(19) United States
(12) Plant Patent Application Publication
(10) Pub. No.: US 2009/0255017 P1
(43) Pub. Date: Oct. 8, 2009

(54) 'Vas-One' Olive tree

(50) ;

(76) Inventors: Elena Sonnoli, Pescia (IT); Alberto Sonnoli, Montecatini Terme (IT); Stefano Sonnoli, Pescia (IT)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 12/378,611

(22) Filed: Feb. 17, 2009

(30) Foreign Application Priority Data
Apr. 4, 2008 (IT) 2008/0715

(51) Int. Cl. A01H 5/00

(52) U.S. Cl. PLT/158

(57) ABSTRACT

The present selection comprises a new and distinct seedling system of olea europaea. This new invention appears to be resistant to pests, diseases and cold temperatures while producing an excellent olive fruit within six to eight years and is primarily grown for olive oil production. The fruit matures in the Tuscany area of Italy in late October to mid-November.

### OLIVE TREE

(olea europaea)

This application claims the benefit of European Union Community Plant Variety Office application designation ITA-01-08, File Number 2008/0175 dated Apr. 4, 2008, a certified copy of which is submitted herewith and incorporated herein in its entirety by this reference.

### BACKGROUND OF THE NEW VARIETY

The present invention relates to a new and distinct variety of olive plant, olea europaea, and will hereinafter be denominated varietally as ‘VAS-ONE’ and more particularly to an olive plant that produces fruit for harvest between late October and mid-November in the Tuscany area of Italy and which is further distinguished by having heavy production and very good oil yield: 60-80% more than the minimum oil of 12% within 6 years, as well as resistance to low temperature. The mother tree is eighteen years old and was discovered in the Uzzand via Darrath-Pistaqa area, Italy with the following dimensions: height 8.2 feet (250 cm) and width 9.84 feet (300 cm). Seedlings were collected from around the mother tree.

This new invention relates to a new and distinct variety of olive tree from a seedling; was selected from another tree in the olive orchard after 10 years of observation; vegetatively propagated in 2008 and exported to the United States. This new variety, ‘VAS-ONE’ has superior production with superior oil quality and prediction.

### ORIGIN AND ASEXUAL PRODUCTION

The new variety has been asexually propagated and produces high oil yields and is somewhat resistant to pests and diseases, and to low temperature and it has very good vigor and high productivity with the plant being suited for olive oil production with the fruit maturing late October to mid-November.

At the site north of Tuscany, after verification of the superior characteristics in production uniformity, olive oil production and quality, vegetatively reproduced cuttings were sent to the U.S.A. in 2008. Upon completion of the post-entry quarantine requirements in California, additional cuttings will be asexually propagated.

### SUMMARY OF THE NEW VARIETY

The new variety of olive was discovered through a selection process from an olive orchard in Tuscany, Italy. Through a selection process beginning in 1998 over a ten year period, the seedling selection process was completed. The new variety is late maturing from late October to mid-November with high productivity and good oil yield within 6-8 years.

### BRIEF DESCRIPTION OF THE DRAWINGS

FIG. 1 displays a view of the new variety tree.

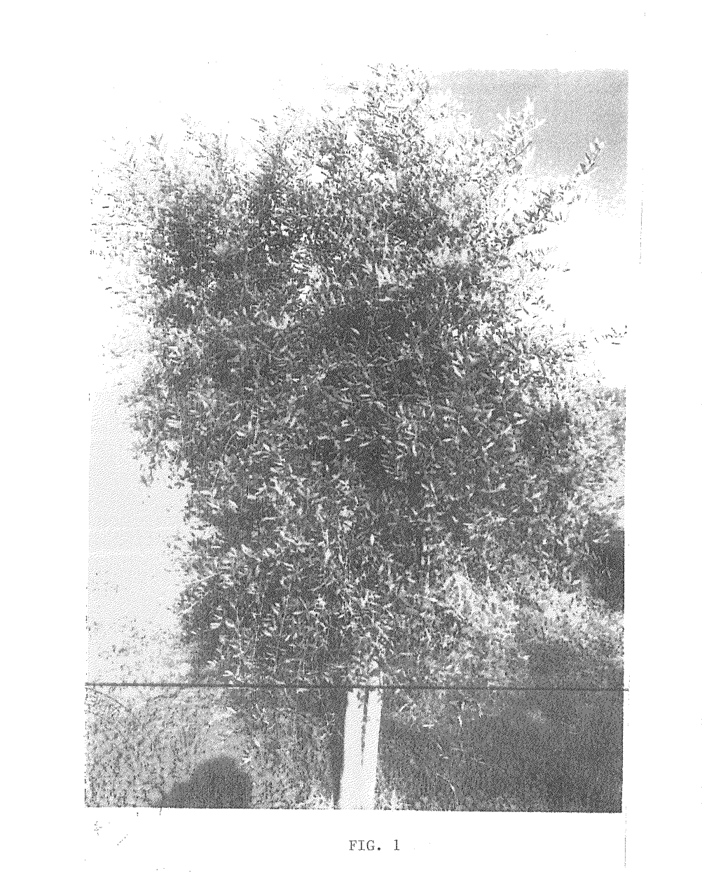

FIG. 2 displays the fruit and fruit clusters along with leaves and young branches.

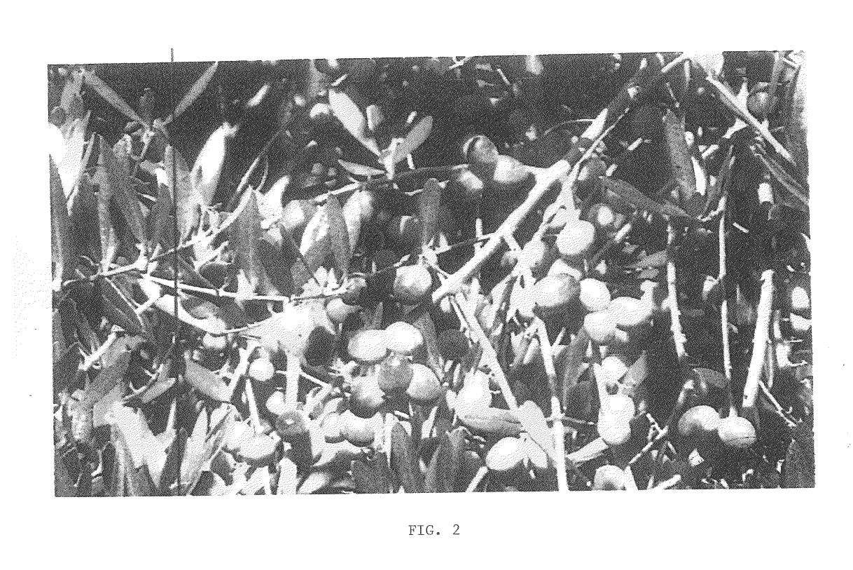

FIG. 3 displays of the mature trunk of the new variety.

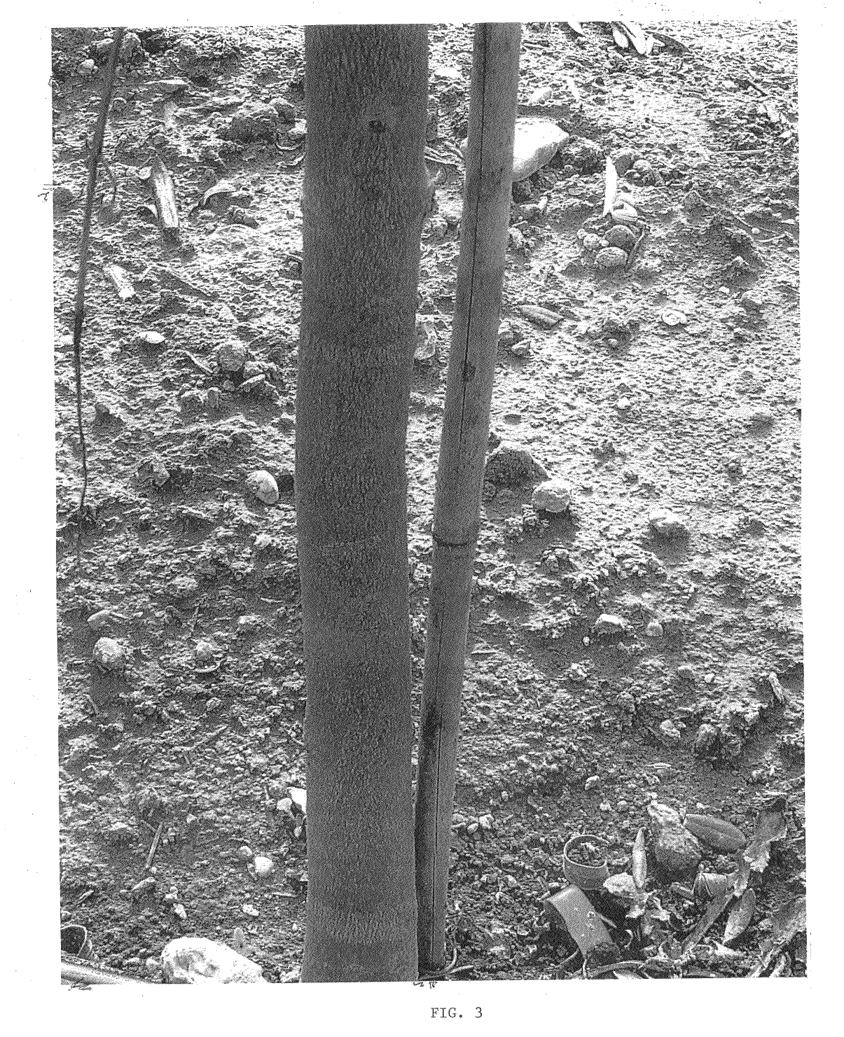

FIG. 4 displays of the two year or older branches with leaves.

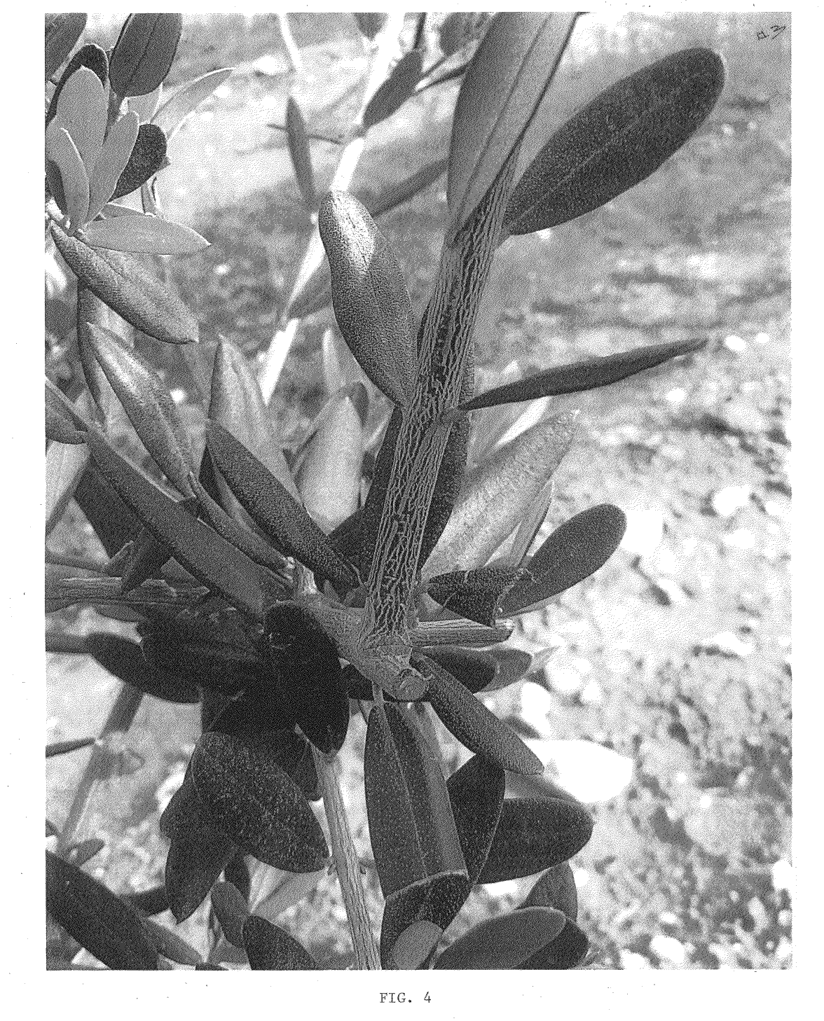

FIG. 5 displays younger branches 1 year old along with older ones.

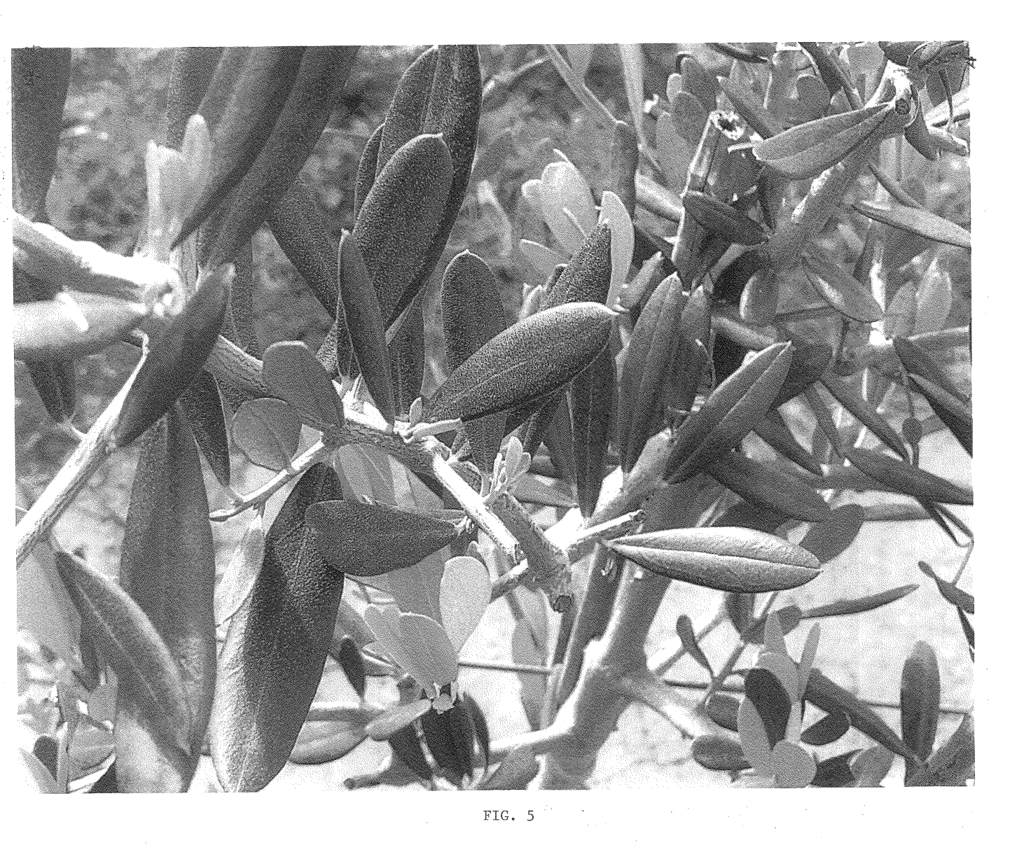

FIG. 6 displays leaves with a mature fruit.

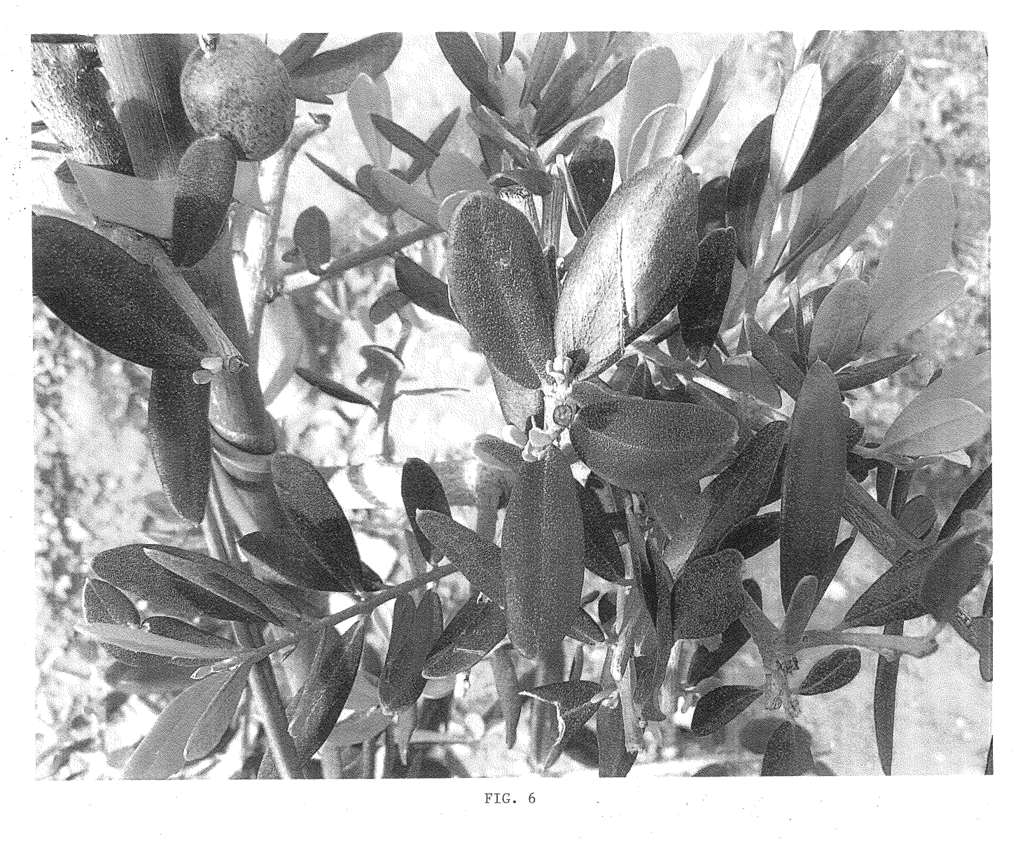

FIG. 7 displays upper leaf and lower leaf surfaces, mature fruit, fruit cut half with seeds and seed.

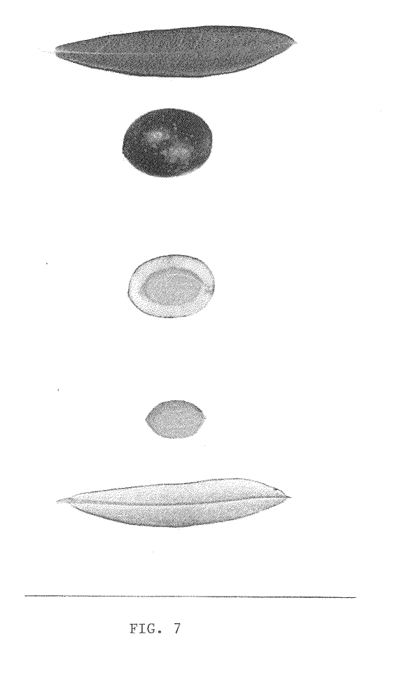

### DETAILED DESCRIPTION

Referring more to the horticultural description and details of the new and distinct variety of olive tree, the following description has been observed under the ecological conditions prevailing in Tuscany, Italy. The age of the observed plant was 6-8 years. All major color designations are by reference to the Dictionary of Color by Maerz & Paul, First Edition, 1930. Common colors are also employed.
Tree: This new invention has medium size trees.
   Height.—8.2 ft. (250 cm).
   Width.—9.84 ft. (300 cm).
   Vigor.—good.
   Chilling requirements.—Normal for olive trees in the Sacramento Valley.
   Figure.—upright and somewhat pyramidal.
   Productivity.—very good.
   Regularity of bearing.—alternate.
Trunk:
   Size.—11.8 ft. (360 cm) circumference.
   Surface texture.—smooth.
   Color code.—Dutch Bl. (44-A-6).
   Branches.—Mature (1 year or older): Size: 0.26 inch (6.5 mm) Immature (less than 1 year): Size: 0.20 inch (5.0 mm) Surface Texture: Mature: smooth Immature: smooth Color: Mature: Light Wedgwood (37-A-7) is the basic color with Slate (39-A-7) as the darker streaked top. Immature: Opaline Gr. (17-A-6) Lenticels: none.
Leaves:
   Leaf size.—small, opposite Length Average — 47.4 mm (1.87 inch) Width Average 12.2 mm (0.48 inch).
   Size.—small, opposite.
   Length.—average 1.87 inch (47.4 mm).
   Width.—average 0.49 inch (12.4 mm).
   Shape.—elliptical.
   Margin.—entire.
   Apex.—obtuse.
   Base.—cuneate.
   Surface texture.—Upper Surface: smooth Downward surface: slightly pubescent.
   Color.—Upward: Polo Green (23-J-9) Downward: Mignon Green (21-J-6).
   Leaf vein.—Color: Mignon Green (21-J-6) Thickness: 1 mm (0.04 in).
   Granular characteristics.—none.
   Petiole.—short, 4-5 mm (0.16-0.20 inch) Thickness: 1-1.5 mm (0.04-0.06 inch) Color: Parrot Green (21-L-6).
   Thickness.—2 mm (0.08 inch).
   Stem glands.—none.
Flower:
   Flower buds.—small, slightly pubescent.
   Average bud size.—small, 2-4 mm (0.08-0.16 inch).
   Date of first bloom.—Mid-May Size: small Petals: 4.
   Number of florets per inflorescence.—10-16.
   Number of sepals.—4.
   Number of petals.—4 Color: white (1-A-1).
   Fragrance.—very mild.
   Reproductive organs.—1 Pistil; 16-27 stamens.
   Pollen color.—Empire Yellow (9-K-3).
Fruit:
   Date of maturity.—late October to mid-November.
   Size.—small.
   Weight.—1.85 grams.
   Diameter.—12-14 mm (0.47-0.55 inch).
   Length.—14-20 mm (0.55-0.79 inch).
   Form.—ovate.
   Suture.—none.
   Stem cavity.—Width: 7 mm (0.28 inch) Depth: average 7 mm (0.28 inch) Length: 7 mm (0.28 inch).
   Stem.—average 4.8 mm (0.19 inch) Caliper: very small.
   Pistil point.—obscure.
   Fruit flesh color.—Pinnard Yellow (9-J-2).
   Fruit surface color.—dark brown, Annapolis (40-C-6).
   Color of pit.—Pinnard Yellow (9-J-2).
Skin:
   Thickness.—less than 1 mm (less than 0.04 inch).
   Texture.—smooth.
   Tendency to crack.—none.
Seed:
   Type.—cling.
   Fibers.—none.
   Size.—Length: approximately 14 mm (0.55 inches) Width: approximately 9 mm (0.35 inches) Thickness: approximately 9 mm (0.35 inches).
   Form.—elliptical.
   Apex.—slightly pointed.
   Color.—Blush Peach Beige (11-A-6).
   Base.—slightly truncate.
   Mucron.—obscure.
   Suture.—slightly marked.
   Sides.—about even.
   Ridges.—faint and uneven.
   Tendency to split.—none known.
   Use.—oil production.
   Resistance.—yes, to pests and diseases.
   Harvesting.—mechanical.
Oil:
   Virgin oil fatty acid percentages.—Miristic Acid: 0.005% Palmitic Acid: 13.112% Palmitoleic Acid: 0.795% Margaric Acid: 0.044% Margaroleic Acid: 0.078% Estearic Acid: 2.465% Oleic Acid: 72.909% Linoleic Acid: 9.034% Araquidic Acid: 0.361% Linolenic Acid: 0.829% Gadoleic Acid: 0.238% Behenic Acid: 0.099% Lignoceric Acid: 0.026%
   M/P ratio (monosaturated fats/polysaturated fats).—5.4.
   Polyphenols (ppm caiteic acid).—400 ppm (mg/Kg).
   K225 (Bitterness).—1.

What is claimed is:

1. A new distinct olive plant as described above with better vigor and higher productivity than ‘Minerva’ with very good fruit production for olive oil within six to eight years with fruit maturing in late October to mid-November in the Tuscany area of Italy.
